(19) United States
(12) Patent Application Publication
(10) Pub. No.: US 2025/0366927 A1
(43) Pub. Date: Dec. 4, 2025

(54) VISUAL GUIDANCE FOR REPOSITIONING A COMPUTER-ASSISTED SYSTEM

(71) Applicant: INTUITIVE SURGICAL OPERATIONS, INC., Sunnyvale, CA (US)

(72) Inventors: Dinesh RABINDRAN, San Jose, CA (US); Simon P. DIMAIO, San Carlos, CA (US); Omid MOHARERI, San Francisco, CA (US)

(21) Appl. No.: 18/874,452

(22) PCT Filed: Jun. 14, 2023
(86) PCT No.: PCT/US2023/025249

§ 371 Date: Dec. 12, 2024

Related U.S. Application Data
(60) Provisional application No. 63/352,594, filed on Jun. 15, 2022.

(51) Int. Cl. A61B 34/20; A61B 34/35; A61B 90/00; A61B 90/50; G16H 40/63

(52) U.S. Cl. CPC A61B 34/20; A61B 34/35; G16H 40/63; A61B 2090/364; A61B 2090/372; A61B 2090/502

(57) ABSTRACT

Techniques for displaying an extended reality (XR) indication of a potential collision between a portion of an object and a portion of a computer-assisted system include the following. The computer-assisted system comprises a sensor system configured to capture sensor data of an environment, and a control system communicably coupled to the sensor system. The control system is configured to: determine a pose of a portion of an object in the environment based on the sensor data, determine a pose of a portion of the computer-assisted system, determine at least one characteristic associated with a potential collision between the portion of the object and the portion of the computer-assisted system, select the potential collision for display based on the at least one characteristic, and cause an XR indication of the potential collision to be displayed to an operator via a display system.

### RELATED APPLICATIONS

This application claims the benefit to U.S. Provisional Application No. 63/352,594, filed Jun. 15, 2022, and entitled “Visual Guidance for Repositioning a Computer-assisted System,” which is incorporated by reference herein.

### TECHNICAL FIELD

The present disclosure relates generally to electronic systems and more particularly relates to visual guidance for repositioning a computer-assisted system.

### BACKGROUND

Computer-assisted electronic systems are being used more and more often. This is especially true in industrial, entertainment, educational, and other settings. As a medical example, the medical facilities of today have large arrays of electronic systems being found in operating rooms, interventional suites, intensive care wards, emergency rooms, and/or the like. Many of these electronic systems may be capable of autonomous or semi-autonomous motion. It is also known for personnel to control the motion and/or operation of electronic systems using one or more input devices located at a user control system. As a specific example, minimally invasive, robotic telesurgical systems permit surgeons to operate on patients from bedside or remote locations. Telesurgery refers generally to surgery performed using surgical systems where the surgeon uses some form of remote control, such as a servomechanism, to manipulate surgical instrument movements rather than directly holding and moving the instruments by hand.

Oftentimes, an electronic system needs to be repositioned within a physical environment in order to give the electronic system access to a worksite. Returning to the medical example, the electronic system may include a medical system that needs to be repositioned to provide access to an interior anatomy of a patient. The physical environment can include obstacles, such as the patient, an operating table, other equipment, fixtures such as lighting fixtures, personnel, and/or the like, that should be avoided when repositioning the medical system. Conventionally, repositioning an electronic system can require a team of two or more operators to communicate verbally and/or through gestures to move the electronic system while avoiding obstacles. However, the operators can be inexperienced or otherwise benefit from assistance to reposition the electronic system properly while avoiding obstacles. In the medical context, observing and reacting to obstacles also distracts from the attention operators may need to pay to other stimuli such as patient status and location, and tasks being performed by others.

Accordingly, improved techniques for assisting operators in repositioning computer-assisted systems are desirable.

### SUMMARY

Consistent with some embodiments, a computer-assisted system includes a sensor system configured to capture sensor data of an environment, and a control system communicably coupled to the sensor system. The control system is configured to: determine a pose of a portion of an object in the environment based on the sensor data, the pose of the portion of the object comprising at least one parameter selected from the group consisting of: a position of the portion of the object and an orientation of the portion of the object, determine a pose of a portion of the computer-assisted system, the pose of the portion of the computer-assisted system comprising at least one parameter selected from the group consisting of: a position of the portion of the computer-assisted system and an orientation of the portion of the computer-assisted system, determine at least one characteristic associated with a potential collision between the portion of the object and the portion of the computer-assisted system based on the pose of the portion of the object and the pose of the portion of the computer-assisted system, select the potential collision for display based on the at least one characteristic, and cause an extended reality (XR) indication of the potential collision to be displayed to an operator via a display system.

Consistent with some embodiments, a method includes determining a pose of a portion of an object in an environment based on sensor data captured by a sensor system, the pose of the portion of the object comprising at least one parameter selected from the group consisting of: a position of the portion of the object and an orientation of the portion of the object. The method further includes determining a pose of a portion of a computer-assisted system, the pose of the portion of the computer-assisted system comprising at least one parameter selected from the group consisting of: a position of the portion of the computer-assisted system and an orientation of the portion of the computer-assisted system. The method also includes determining at least one characteristic associated with a potential collision between the portion of the object and the portion of the computer-assisted system based on the pose of the portion of the object and the pose of the portion of the computer-assisted system. In addition, the method includes selecting the potential collision for display based on the at least one characteristic, and causing an extended reality (XR) indication of the potential collision to be displayed to an operator via a display system.

Other embodiments include, without limitation, one or more non-transitory machine-readable media including a plurality of machine-readable instructions, which when executed by one or more processors, are adapted to cause the one or more processors to perform any of the methods disclosed herein.

Other embodiments include, without limitation, one or more non-transitory machine-readable media including a plurality of machine-readable instructions, which when executed by one or more processors, are adapted to cause the one or more processors to perform any of the methods disclosed herein.

The foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTION OF THE DRAWINGS

FIG. 1 is a simplified diagram including an example of a computer-assisted system, according to various embodiments.

FIG. 2 depicts an illustrative configuration of a sensor system for use with a computer-assisted system, according to various embodiments.

FIG. 3 depicts an illustrative configuration of a display system for use with a computer-assisted system, according to various embodiments.

FIG. 4 illustrates the control module of FIG. 1 in greater detail, according to various embodiments.

FIG. 5 illustrates a simplified diagram of a method for providing visual guidance for repositioning a computer-assisted system, according to various embodiments.

FIG. 6 illustrates in greater detail a process of the method of FIG. 5 for determining one or more characteristics of each potential collision, according to various embodiments.

FIG. 7 illustrates in greater detail a process of the method of FIG. 5 for determining one or more characteristics of each potential collision, according to other various embodiments.

FIG. 8 illustrates in greater detail a process of the method of FIG. 5 for causing an extended reality indication to be displayed, according to various embodiments.

FIG. 9 illustrates in greater detail a process of the method of FIG. 5 for causing an extended reality indication to be displayed, according to other various embodiments.

FIGS. 10A-10B illustrates example two-dimensional visual guidance displays, according to various embodiments.

FIG. 11 illustrates an example three-dimensional visual guidance display, according to various embodiments.

### DETAILED DESCRIPTION

This description and the accompanying drawings that illustrate inventive aspects, embodiments, embodiments, or modules should not be taken as limiting-the claims define the protected invention. Various mechanical, compositional, structural, electrical, and operational changes may be made without departing from the spirit and scope of this description and the claims. In some instances, well-known circuits, structures, or techniques have not been shown or described in detail in order not to obscure the invention. Like numbers in two or more figures represent the same or similar elements.

In this description, specific details are set forth describing some embodiments consistent with the present disclosure. Numerous specific details are set forth in order to provide a thorough understanding of the embodiments. It will be apparent, however, to one skilled in the art that some embodiments may be practiced without some or all of these specific details. The specific embodiments disclosed herein are meant to be illustrative but not limiting. One skilled in the art may realize other elements that, although not specifically described here, are within the scope and the spirit of this disclosure. In addition, to avoid unnecessary repetition, one or more features shown and described in association with one embodiment may be incorporated into other embodiments unless specifically described otherwise or if the one or more features would make an embodiment non-functional.

Further, the terminology in this description is not intended to limit the invention. For example, spatially relative terms-such as “beneath”, “below”, “lower”, “above”, “upper”, “proximal”, “distal”, and the like-may be used to describe one element's or feature's relationship to another element or feature as illustrated in the figures. These spatially relative terms are intended to encompass different positions (i.e., locations) and orientations (i.e., rotational placements) of the elements or their operation in addition to the position and orientation shown in the figures. For example, if the content of one of the figures is turned over, elements described as “below” or “beneath” other elements or features would then be “above” or “over” the other elements or features. Thus, the exemplary term “below” can encompass both positions and orientations of above and below. A device may be otherwise oriented (rotated 90 degrees or at other orientations) and the spatially relative descriptors used herein interpreted accordingly. Likewise, descriptions of movement along and around various axes include various special element positions and orientations. In addition, the singular forms “a”, “an”, and “the” are intended to include the plural forms as well, unless the context indicates otherwise. And, the terms “comprises”, “comprising”, “includes”, and the like specify the presence of stated features, steps, operations, elements, and/or components but do not preclude the presence or addition of one or more other features, steps, operations, elements, components, and/or groups. Components described as coupled may be electrically or mechanically directly coupled, or they may be indirectly coupled via one or more intermediate components.

Elements described in detail with reference to one embodiment, embodiment, or module may, whenever practical, be included in other embodiments, embodiments, or modules in which they are not specifically shown or described. For example, if an element is described in detail with reference to one embodiment and is not described with reference to a second embodiment, the element may nevertheless be claimed as included in the second embodiment. Thus, to avoid unnecessary repetition in the following description, one or more elements shown and described in association with one embodiment, embodiment, or application may be incorporated into other embodiments, embodiments, or aspects unless specifically described otherwise, unless the one or more elements would make an embodiment or embodiment non-functional, or unless two or more of the elements provide conflicting functions.

In some instances, well known methods, procedures, components, and circuits have not been described in detail so as not to unnecessarily obscure aspects of the embodiments.

This disclosure describes various elements (such as systems and devices, and portions of systems and devices) in three-dimensional space. As used herein, the term “position” refers to the location of an element or a portion of an element in a three-dimensional space (e.g., three degrees of translational freedom along Cartesian x-, y-, and z-coordinates). As used herein, the term “orientation” refers to the rotational placement of an element or a portion of an element (three degrees of rotational freedom-e.g., roll, pitch, and yaw). As used herein, the term “pose” refers to the multi-degree of freedom (DOF) spatial position and/or orientation of a coordinate system of interest attached to a rigid body. In general, a pose can include a pose variable for each of the DOFs in the pose. For example, a full 6-DOF pose would include 6 pose variables corresponding to the 3 positional DOFs (e.g., x, y, and z) and the 3 orientational DOFs (e.g., roll, pitch, and yaw). A 3-DOF position only pose would include only pose variables for the 3 positional DOFs. Similarly, a 3-DOF orientation only pose would include only pose variables for the 3 rotational DOFs. Poses with any other number of DOFs (e.g., one, two, four, or five) are also possible. As used herein, the term “shape” refers to a set positions or orientations measured along an element. As used herein, and for an element or portion of an element, e.g. a device (e.g., a computer-assisted system or a repositionable arm), the term “proximal” refers to a direction toward the base of the system or device of the repositionable arm along its kinematic chain, and the term “distal” refers to a direction away from the base along the kinematic chain.

Aspects of this disclosure are described in reference to computer-assisted systems, which may include systems and devices that are teleoperated, remote-controlled, autonomous, semiautonomous, manually manipulated, and/or the like. Example computer-assisted systems include those that comprise robots or robotic devices. Further, aspects of this disclosure are described in terms of an embodiment using a medical system, such as the da Vinci® Surgical System commercialized by Intuitive Surgical, Inc. of Sunnyvale, California. Knowledgeable persons will understand, however, that inventive aspects disclosed herein may be embodied and implemented in various ways, including robotic and, if applicable, non-robotic embodiments. Embodiments described for da Vinci® Surgical Systems are merely exemplary, and are not to be considered as limiting the scope of the inventive aspects disclosed herein. For example, techniques described with reference to surgical instruments and surgical methods may be used in other contexts. Thus, the instruments, systems, and methods described herein may be used for humans, animals, portions of human or animal anatomy, industrial systems, general robotic, or teleoperational systems. As further examples, the instruments, systems, and methods described herein may be used for non-medical purposes including industrial uses, general robotic uses, sensing or manipulating non-tissue work pieces, cosmetic improvements, imaging of human or animal anatomy, gathering data from human or animal anatomy, setting up or taking down systems, training medical or non-medical personnel, and/or the like. Additional example applications include use for procedures on tissue removed from human or animal anatomies (with or without return to a human or animal anatomy) and for procedures on human or animal cadavers. Further, these techniques can also be used for medical treatment or diagnosis procedures that include, or do not include, surgical aspects.

### System Overview

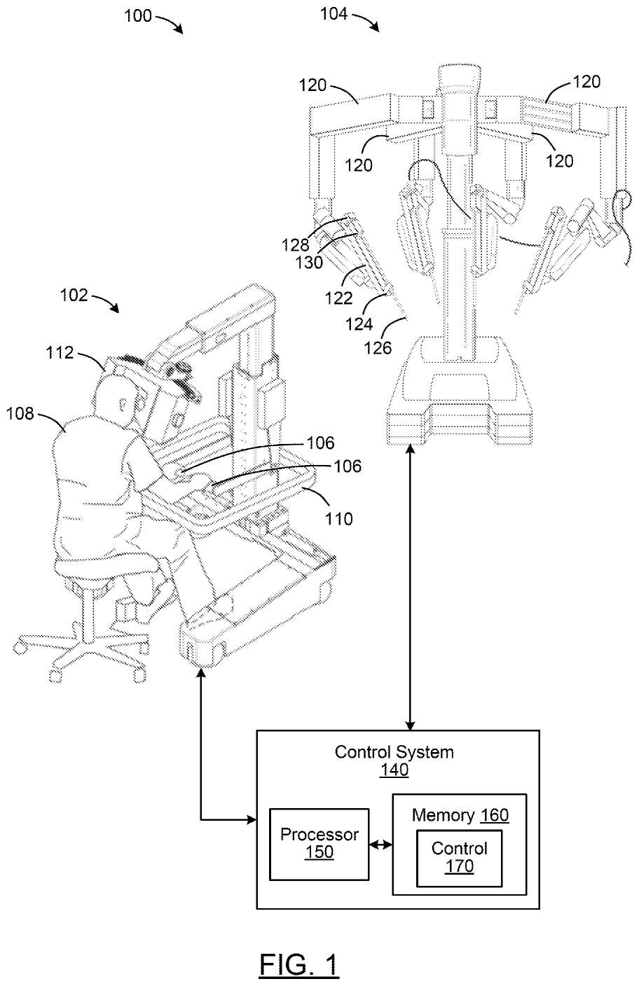

FIG. 1 is a simplified diagram of an example computer-assisted system 100, according to various embodiments. In some examples, the computer-assisted system 100 is a teleoperated system. In medical examples, computer-assisted system 100 can be a teleoperated medical system such as a surgical system. As shown, computer-assisted system 100 includes a follower device 104 that can be teleoperated by being controlled by one or more leader devices (also called “leader input devices” when designed to accept external input), described in greater detail below. Systems that include a leader device and a follower device are referred to as leader-follower systems, and also sometimes referred to as master-slave systems. Also shown in FIG. 1 is an input system that includes a workstation 102 (e.g., a console), and in various embodiments the input system can be in any appropriate form and may or may not include a workstation 102.

In the example of FIG. 1, workstation 102 includes one or more leader input devices 106 that are designed to be contacted and manipulated by an operator 108. For example, workstation 102 can comprise one or more leader input devices 106 for use by the hands, the head, or some other body part(s) of operator 108. Leader input devices 106 in this example are supported by workstation 102 and can be mechanically grounded. In some embodiments, an ergonomic support 110 (e.g., forearm rest) can be provided on which operator 108 can rest his or her forearms. In some examples, operator 108 can perform tasks at a worksite near follower device 104 during a procedure by commanding follower device 104 using leader input devices 106.

A display unit 112 is also included in workstation 102. Display unit 112 can display images for viewing by operator 108. Display unit 112 can be moved in various degrees of freedom to accommodate the viewing position of operator 108 and/or to optionally provide control functions as another leader input device. In the example of computer-assisted system 100, displayed images can depict a worksite at which operator 108 is performing various tasks by manipulating leader input devices 106 and/or display unit 112. In some examples, images displayed by display unit 112 can be received by workstation 102 from one or more imaging devices arranged at a worksite. In other examples, the images displayed by display unit 112 can be generated by display unit 112 (or by a different connected device or system), such as for virtual representations of tools, the worksite, or for user interface components.

When using workstation 102, operator 108 can sit in a chair or other support in front of workstation 102, position his or her eyes in front of display unit 112, manipulate leader input devices 106, and rest his or her forearms on ergonomic support 110 as desired. In some embodiments, operator 108 can stand at the workstation or assume other poses, and display unit 112 and leader input devices 106 can be adjusted in position (height, depth, etc.) to accommodate operator 108.

In some embodiments, the one or more leader input devices 106 can be ungrounded (ungrounded leader input devices being not kinematically grounded, such as leader input devices held by the hands of operator 108 without additional physical support). Such ungrounded leader input devices can be used in conjunction with display unit 112. In some embodiments, operator 108 can use a display unit 112 positioned near the worksite, such that operator 108 manually operates instruments at the worksite, such as a laparoscopic instrument in a surgical example, while viewing images displayed by display unit 112.

Computer-assisted system 100 can also include follower device 104, which can be commanded by workstation 102. In a medical example, follower device 104 can be located near an operating table (e.g., a table, bed, or other support) on which a patient can be positioned. In some medical examples, the worksite is provided on an operating table, e.g., on or in a patient, simulated patient, or model, etc. (not shown). The follower device 104 shown includes a plurality of manipulator arms 120, each manipulator arm 120 configured to couple to an instrument assembly 122. An instrument assembly 122 can include, for example, an instrument 126. As shown, each instrument assembly 122 is mounted to a distal portion of a respective manipulator arm 120. The distal portion of each manipulator arm 120 further includes a cannula mount 124 which is configured to have a cannula (not shown) mounted thereto. When a cannula is mounted to the cannula mount, a shaft of an instrument 126 passes through the cannula and into a worksite, such as a surgery site during a surgical procedure. The distal portion of each manipulator arm 120 further includes a cannula mount 124 which is configured to have a cannula (not shown) mounted thereto. When a cannula is mounted to the cannula mount, a shaft of an instrument 126 passes through the cannula and into a worksite, such as a surgery site during a surgical procedure. A force transmission mechanism 130 of the instrument assembly 122 can be connected to an actuation interface assembly 128 of the manipulator arm 120 that includes drive and/or other mechanisms controllable from workstation 102 to transmit forces to the force transmission mechanism 130 to actuate the instrument 126.

In various embodiments, one or more of instruments 126 can include an imaging device for capturing images (e.g., optical cameras, hyperspectral cameras, ultrasonic sensors, etc.). For example, one or more of instruments 126 can be an endoscope assembly that includes an imaging device, which can provide captured images of a portion of the worksite to be displayed via display unit 112.

In some embodiments, the manipulator arms 120 and/or instrument assemblies 122 can be controlled to move and articulate instruments 126 in response to manipulation of leader input devices 106 by operator 108, and in this way “follow” the leader input devices 106 through teleoperation. This enables the operator 108 to perform tasks at the worksite using the manipulator arms 120 and/or instrument assemblies 122. Manipulator arms 120 are examples of repositionable structures that a computer-assisted device (e.g., follower device 104) can include. In some embodiments, a repositionable structure of a computer-assisted device can include a plurality of links that are rigid members and joints that are movable components that can be actuated to cause relative motion between adjacent links. For a surgical example, the operator 108 can direct follower manipulator arms 120 to move instruments 126 to perform surgical procedures at internal surgical sites through minimally invasive apertures or natural orifices.

As shown, a control system 140 is provided external to workstation 102 and communicates with workstation 102. In other embodiments, control system 140 can be provided in workstation 102 or in follower device 104. As operator 108 moves leader input device(s) 106, sensed spatial information including sensed position and/or orientation information is provided to control system 140 based on the movement of leader input devices 106. Control system 140 can determine or provide control signals to follower device 104 to control the movement of manipulator arms 120, instrument assemblies 122, and/or instruments 126 based on the received information and operator input. In one embodiment, control system 140 supports one or more wired communication protocols, (e.g., Ethernet, USB, and/or the like) and/or one or more wireless communication protocols (e.g., Bluetooth, IrDA, HomeRF, IEEE 1102.11, DECT, Wireless Telemetry, and/or the like).

Control system 140 can be implemented on one or more computing systems. One or more computing systems can be used to control follower device 104. In addition, one or more computing systems can be used to control components of workstation 102, such as movement of a display unit 112.

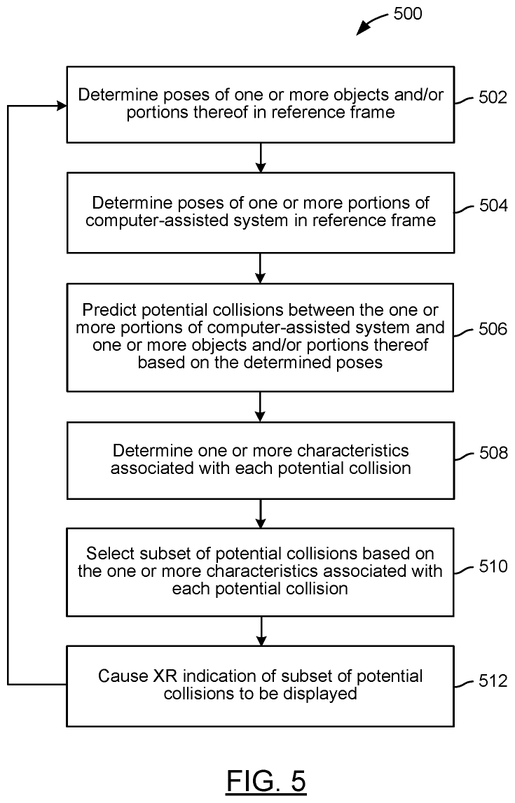

As shown, control system 140 includes a processor 150 and a memory 160 storing a control module 170. In some embodiments, control system 140 can include one or more processors, non-persistent storage (e.g., volatile memory, such as random access memory (RAM), cache memory), persistent storage (e.g., a hard disk, an optical drive such as a compact disk (CD) drive or digital versatile disk (DVD) drive, a flash memory, a floppy disk, a flexible disk, a magnetic tape, any other magnetic medium, any other optical medium, programmable read-only memory (PROM), an erasable programmable read-only memory (EPROM), a FLASH-EPROM, any other memory chip or cartridge, punch cards, paper tape, any other physical medium with patterns of holes, etc.), a communication interface (e.g., Bluetooth interface, infrared interface, network interface, optical interface, etc.), and numerous other elements and functionalities. The non-persistent storage and persistent storage are examples of non-transitory, tangible machine readable media that can include executable code that, when run by one or more processors (e.g., processor 150), can cause the one or more processors to perform one or more of the techniques disclosed herein, including the processes of method 500 and/or the processes of FIGS. 5-9, described below. In addition, functionality of control module 170 can be implemented in any technically feasible software and/or hardware in some embodiments.

Each of the one or more processors of control system 140 can be an integrated circuit for processing instructions. For example, the one or more processors can be one or more cores or micro-cores of a processor, a central processing unit (CPU), a microprocessor, a field-programmable gate array (FPGA), an application-specific integrated circuit (ASIC), a digital signal processor (DSP), a graphics processing unit (GPU), a tensor processing unit (TPU), and/or the like. Control system 140 can also include one or more input devices, such as a touchscreen, keyboard, mouse, microphone, touchpad, electronic pen, or any other type of input device.

A communication interface of control system 140 can include an integrated circuit for connecting the computing system to a network (not shown) (e.g., a local area network (LAN), a wide area network (WAN) such as the Internet, mobile network, or any other type of network) and/or to another device, such as another computing system.

Further, control system 140 can include one or more output devices, such as a display device (e.g., a liquid crystal display (LCD), a plasma display, touchscreen, organic LED display (OLED), projector, or other display device), a printer, a speaker, external storage, or any other output device. One or more of the output devices can be the same or different from the input device(s). Many different types of computing systems exist, and the aforementioned input and output device(s) can take other forms.

In some embodiments, control system 140 can be connected to or be a part of a network. The network can include multiple nodes. Control system 140 can be implemented on one node or on a group of nodes. By way of example, control system 140 can be implemented on a node of a distributed system that is connected to other nodes. By way of another example, control system 140 can be implemented on a distributed computing system having multiple nodes, where different functions and/or components of control system 140 can be located on a different node within the distributed computing system. Further, one or more elements of the aforementioned control system 140 can be located at a remote location and connected to the other elements over a network.

Some embodiments can include one or more components of a teleoperated medical system such as a da Vinci® Surgical System, commercialized by Intuitive Surgical, Inc. of Sunnyvale, California, U.S.A. Embodiments on da Vinci® Surgical Systems are merely examples and are not to be considered as limiting the scope of the features disclosed herein. For example, different types of teleoperated systems having follower devices at worksites, as well as non-teleoperated systems, can make use of features described herein.

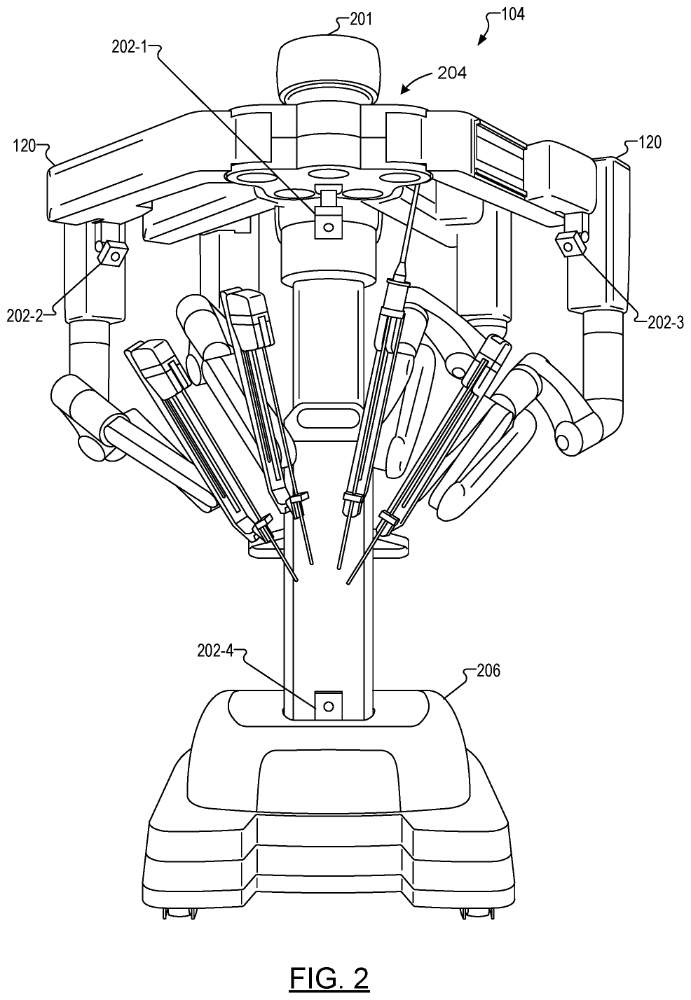

FIG. 2 depicts an illustrative configuration of a sensor system, according to various embodiments. As shown, imaging devices 202 (imaging devices 202-1 through 202-4) are attached to portions of follower device 104. Although described herein with respect to imaging devices as a reference example, in some embodiments, a sensor system can include any technically feasible sensors, such as monoscopic and stereoscopic optical systems, ultrasonic systems, depth cameras such as cameras using time-of-flight sensors, LIDAR (light detection and ranging) sensors, etc. that are mounted on a computer-assisted system and/or elsewhere. For example, one or more sensors can be mounted on a base, on an orienting platform 204, and/or on one or more manipulator arms 120 of follower device 104. As another example, one or more sensors can be worn by an operator or mounted to a wall, a ceiling, the floor, or other equipment such as tables or carts.

Illustratively, imaging device 202-1 is attached to orienting platform 204 of follower device 104, imaging device 202-2 is attached to manipulating arm 120-1 of follower device 104, imaging device 202-3 is attached to manipulating arm 120-4 of follower device 104, and imaging device 202-4 is attached to a base 206 of follower device 104. In implementations in which follower device 104 is positioned proximate to a patient (e.g., as a patient side cart), placement of imaging devices 202 at strategic locations on follower device 104 provides advantageous imaging viewpoints proximate to a patient and areas around a worksite where a surgical procedure is to be performed on the patient.

The placements of imaging devices 202 on components of follower device 104 as shown in FIG. 2 are illustrative. Additional and/or alternative placements of any suitable number of imaging devices 202 and/or other sensors on follower device 104, other components of computer-assisted system 100, and/or other components (not shown) located in proximity to the follower device 104 can be used in sensor systems in other embodiments. Imaging devices 202 and/or other sensors can be attached to components of follower device 104, other components of computer-assisted system 100, and/or other components in proximity to follower device 104 in any suitable way. Additional computer-assisted systems including sensor systems that include sensors are described in International Application Publication No. WO 2021/097332, filed Nov. 13, 2020, and titled “Visibility Metrics in Multi-View Medical Activity Recognition Systems and Methods,” which is hereby incorporated by reference herein.

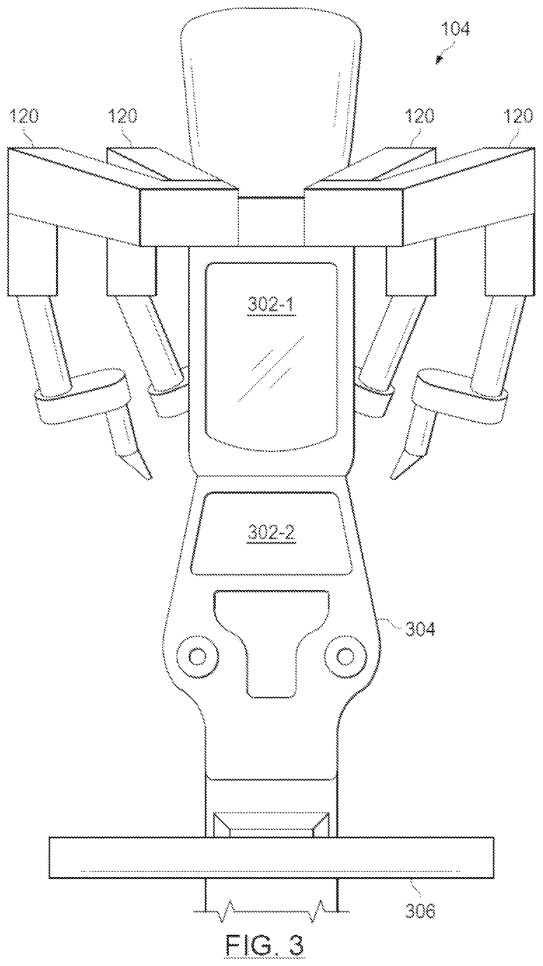

FIG. 3 depicts an illustrative configuration of a display system, according to various embodiments. As shown, a user control interface (helm) 304 of follower device 104 includes display devices 302 (display devices 302-1 and 302-2). Illustratively, the user control interface 304 is attached to a repositionable structure of follower device 104 on a side opposite from manipulator arms 120. Display devices 302 are example output devices of follower device 104. In some embodiments, follower device 104 can include any technically feasible output device or devices. For example, one or more of display devices 302 can be cathode-ray tube (CRT) devices, liquid crystal display (LCD) devices, light-emitting diode (LED) devices, organic light-emitting diode (OLED) devices, quantum dot light-emitting diode (QLED) devices, plasma display devices, touchscreens, projectors, etc.

Illustratively, user control interface (helm) 304 also includes handlebars 306 that an operator can push or pull to reposition follower device 104 within an environment. In some embodiments, follower device 104 includes one or more actuators (e.g., one or more electric motors or servos) that drive the wheels (not shown) of follower device 104 based on input from the operator to assist an operator in repositioning follower device 104. For example, forces or torques applied by the operator on handlebars 306 can be used to determine a direction and speed of the one or more actuators. In some examples, user control interface 304 can include one or more buttons or other input devices (e.g., a joystick) to provide directional commands for controlling the one or more actuators. In some embodiments, repositioning of follower device 104 can be semi-autonomous or fully autonomous. In some other embodiments, follower device 104 does not include one or more actuators that assist the operator in repositioning follower device 104.

The placements of display devices 302 on follower device 104 as shown in FIG. 3 are illustrative. Additional and/or alternative placements of any suitable number of display devices 302 on follower device 104, other components of computer-assisted system 100, and/or other components (not shown) located in proximity to follower device 104 can be used in other embodiments. For example, one or more display devices can be attached to components of follower device 104, other components of computer-assisted system 100, and/or other components in proximity to follower device 104 in any suitable way. As further examples, one or more display devices can be included in a handheld device or a head-mounted device.

### Visual Guidance for Repositioning a Computer-Assisted System

A computer-assisted system can be repositioned within a physical environment while reducing the risk of collisions with obstacles. In some embodiments, repositioning the computer-assisted system includes generating and displaying extended reality (XR) indications of potential collisions between portions of a computer-assisted system and portions of objects in a physical environment.

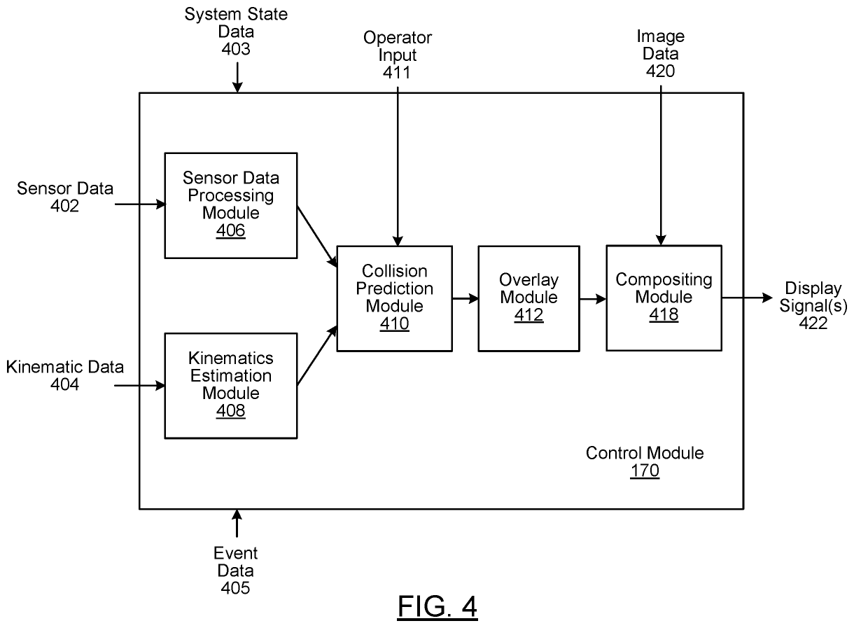

FIG. 4 illustrates control module 170 of FIG. 1 in greater detail, according to various embodiments. As shown, control module 170 includes a sensor data processing module 406, a kinematics estimation module 408, a collision prediction module 410, an overlay module 412, and a compositing module 418. Sensor data processing module 406 receives sensor data 402 and determines the poses of objects, and/or portions thereof, based on sensor data 402. As used herein, a pose can include a position and/or an orientation.

Examples of sensor data 402 and sensor(s) for collecting sensor data 402 are described above in conjunction with FIG. 2. Examples of objects and/or portions of objects in the medical context include a patient, a profile of a patient, an operator, other personnel, a cannula, a fixture, an operating table, equipment (e.g., stands, patient monitoring equipment, drug delivery systems, imaging systems, patient monitors, etc.), surgical robots and/or accessories, laparoscopic or open-surgery instruments, other obstacles, etc. and/or portions thereof that are in the field of view of one or more sensors. In some examples, objects and/or portions thereof might be in a direction of motion of follower device 104. In some embodiments, sensor data processing module 406 can employ point cloud processing algorithms, object detection, object segmentation, classical computer vision techniques for part/object detection, and/or part segmentation techniques to determine the poses of objects and/or portions thereof. In some embodiments, objects and/or portions of objects can be outside the field of view of the sensor(s). In such cases, techniques known in the art, such simultaneous localization and mapping (SLAM), can be employed to determine the objects and/or portions thereof in a reference frame associated with the sensor(s). Additional and/or alternative techniques for detecting objects and/or portions thereof using sensors are described in International Publication No. WO 2022/104118, filed Nov. 12, 2021, and titled “Visibility Metrics in Multi-view Medical Activity Recognition Systems and Methods,” U.S. Provisional Patent Application No. 63/141,830, filed Jan. 26, 2021, and titled “Scene Perception Systems and Methods,” and International Publication No. WO 2022/104129, filed Nov. 12, 2022, and titled “Multi-view Medical Activity Recognition Systems and Methods,” which are hereby incorporated by reference herein.

Kinematics estimation module 408 receives kinematics data 404 associated with the joints and/or links of a repositionable structure of follower device 104. Given kinematics data 404, kinematics estimation module 408 uses one or more kinematic models of a repositionable structure of follower device 104, and optionally a three-dimensional (3D) model of the computer-assisted system, to determine poses of one or more portions of the computer-assisted system. Returning to the medical example, the poses of portion(s) of follower device 104 can include the heights of a distal portions of manipulator arms (e.g., cannula mounts 124 or instruments 126) and/or other portions of follower device 104, an overall height of follower device 104, horizontal positions of manipulator arms 120 or other portions of follower device 104, orientations of manipulator arms 120 or other portions of follower device 104, and/or the like. In some embodiments, kinematics data 404 is synchronized with sensor data 402 so that comparisons can be made between poses that are determined using both types of data corresponding to the same point in time. In some embodiments, the kinematics data 404 and sensor data 402 are transformed using well-known techniques to a common reference frame. In some examples, the common reference frame is a base reference frame of the repositionable structure. Additional and/or alternative techniques for transforming kinematics and sensor data to a common reference frame, which is also referred to herein as “registering” the follower device and sensor(s) relative to each other, are described in U.S. Provisional Patent Application No. 63/312,765, filed Feb. 22, 2022, and titled “Techniques for Repositioning a Computer-Assisted System with Motion Partitioning,” which is hereby incorporated by reference herein.

Collision prediction module 410 receives the poses of objects and/or portions thereof from sensor data processing module 406 and the poses of portions of the computer-assisted system from kinematics estimation module 408. Collision prediction module 410 makes online predictions in real-time, based on the received poses, of potential collisions between portions of objects and portions of follower device 104, assuming that follower device 104 (and the repositionable structure of follower device 104) continues to move according to a current trajectory. In addition, collision prediction module 410 selects a subset of the potential collisions for display to an operator based on one or more characteristics associated with potential collisions in the subset. In some embodiments, collision prediction module 410 can account for operator preferences, shown as operator input 411. For example, an operator can indicate (e.g., via a touchscreen or other input device) a target object for collision avoidance, a target position to achieve, a reference frame to use, etc. Overlay module 412 generates XR content that includes one or more indications of the subset of potential collisions. For example, the XR content can include augmented reality (AR), mixed reality (MR), and/or virtual reality (VR) content. As used herein, AR refers to a view of the physical environment with an overlay of one or more computer-generated graphical elements, MR refers to an AR environment in which physical objects and computer-generated elements can interact, and VR refers to a virtual environment that includes computer-generated elements.

Compositing module 418 transforms the XR content that is generated to a perspective associated with the view of an imaging device that captures image data 420. Image data 420 can also be included in sensor data 402 or separate from sensor data 402. In addition, compositing module 418 combines the transformed XR content with image data 420 to generate a composite image. Thereafter, compositing module 418 outputs a display signal 422 that can be used to display the composite image. Although described herein primarily with respect to generating a composite image by combining XR content with image data 402, in some embodiments, a perspective-corrected view of the XR content can be displayed without combining the XR content with other image data.

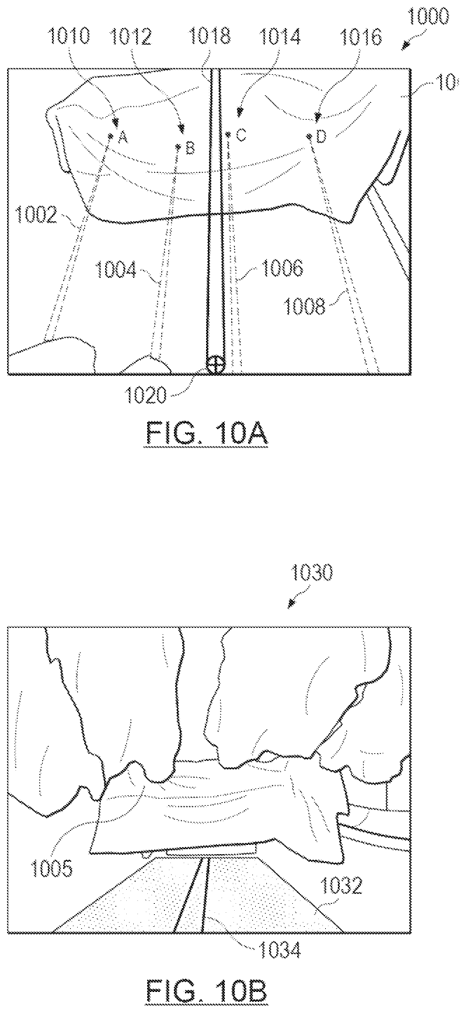

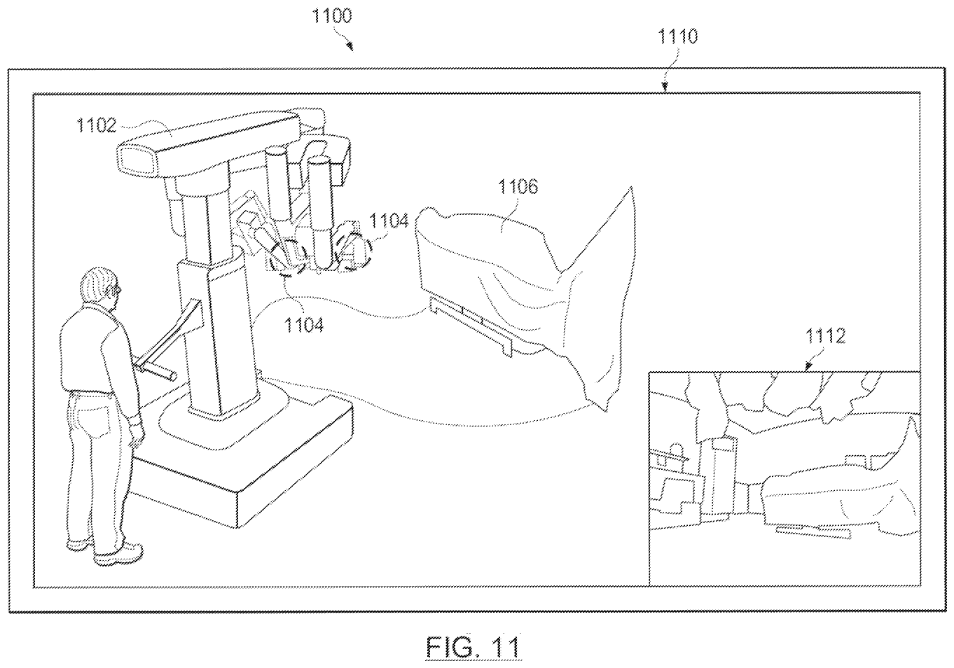

Techniques for predicting potential collisions, selecting the subset of potential collisions, and generating and displaying XR content, according to some embodiments, are discussed in greater detail in conjunction with FIGS. 5-9. Example 2D and 3D visual guidance displays that include XR content, according to some embodiments, are discussed in greater detail below in conjunction with FIGS. 10A-10B and 11, respectively.

In some embodiments, the above behaviors of sensor data processing module 406, kinematics estimation module 408, collision prediction module 410, overlay module 412, and/or compositing module 414 can be allowed, inhibited, stopped, overridden, and/or modified in any technically feasible manner. In some embodiments, control module 170 can receive system state data 403 and/or event data 405 that changes the behaviors of sensor data processing module 406, kinematics estimation module 408, collision prediction module 410, overlay module 412, and/or compositing module 414. For example, system state data 403 can indicate a system mode change that is triggered by entering a certain zone (e.g., a zone that is a given radius around a worksite, a cylindrical zone, a rectangular zone, a zone of irregular shape, etc.), and a different subset of potential collisions can be selected for display as XR content given the system mode change. As another example, system state data 403 and/or event data 405 can cause XR content having different appearances and/or at different locations to be displayed. As yet another example, event data 405 can include data associated with operator interactions that causes XR content having different apprearances and/or at different locations to be displayed. In such cases, the operator can activate/deactivate or modify the XR content, and/or specific XR indications by providing one or more hand inputs that are detected by one or more hand-input sensors (e.g., touch-controlled sensors, gesture-based sensors, switches, etc.), one or more inputs using a user interface, one or more audio commands, etc.

FIG. 5 illustrates a simplified diagram of a method 500 that includes example processes 502-512 for providing visual guidance for repositioning a computer-assisted system, according to various embodiments. In some embodiments, processes 502-512 of method 500 can be performed in real time as a computer-assisted system (e.g., follower device 104) is being repositioned. One or more of the processes 502-512 of method 500 can be implemented, at least in part, in the form of executable code stored on non-transitory, tangible, machine readable media that when run by one or more processors (e.g., the processor 150 in control system 140) cause the one or more processors to perform one or more of the processes 502-512. In some embodiments, method 500 can be performed by one or more modules, such as control module 170. In some embodiments, method 500 can include fewer processes or additional processes, which are not shown. In some embodiments, one or more of the processes 502-512 can be ordered differently than shown in FIG. 5. In some embodiments, one or more of the processes 502-512 can be combined or performed simultaneously. In some embodiments, one or more of the processes 502-512 can be performed, at least in part, by one or more of the modules of control system 140.

As shown, method 500 begins at process 502, where the poses of one or more portions of objects are determined in a reference frame. In some embodiments, the objects and/or portions thereof can be selected in any technically feasible manner. In some embodiments, the objects and/or portions thereof can be selected based on defaults, an operating procedure, operator preference, whether an object is in the current field of view of the operator, a configuration/speed/direction of the computer-assisted system (e.g., follower device 104), etc. As another example, in some embodiments, portions of objects can be obtained by dividing each selected object into portions using a grid, a quad tree, an octree, and/or other partitioning mechanisms. In some embodiments, the reference frame can be a global reference frame. In some embodiments, the reference frame can be attached to the computer-assisted system or a portion thereof, such as a particular sensor. In some embodiments, the reference frame to use can be selected by an operator via a user interface, an input device, voice command, or the like.

In some embodiments, poses of the one or more portions of objects are determined at process 502 based on sensor data and a machine learning or other computer vision technique. For example, point cloud processing algorithms, object detection, object segmentation, and/or part segmentation techniques can be used to determine the poses of objects and/or portions thereof. As a specific example, a machine learning model, such as a convolutional neural network, can be trained to recognize objects and/or portions thereof in sensor data, as well as to estimate the poses of those objects and/or portions thereof. As another example, a computer vision technique that employs hand-coded features can be used to recognize objects and/or portions thereof and to estimate the poses of those objects and/or portions thereof. As yet another example, the poses of objects and/or portions thereof can be determined using a combination of deep learning models and point cloud processing algorithms. In such a case, the deep learning model can segment the objects and/or portions thereof, and the point cloud processing algorithms can determine the boundaries and/or poses of those objects and/or portions thereof. In addition, the objects and the computer-assisted system (and/or portions thereof) can be registered to one another based on image data depicting the poses of the object and the computer-assisted system, laser ranging data, ultrasonic data, RFID or emitter-receiver data usable for locating or orienting components relative to each other, and/or based on any other suitable data. As described above in conjunction with FIG. 4, the registration establishes a relationship between the object and the computer-assisted system (and/or the portions thereof) so that the poses of portions of the objects and/or portions thereof can be determined relative to portions of the computer-assisted system. In some examples, the registration relationship is a six degrees of freedom pose. In some embodiments, the registration relationship can be determined based on the known position and orientation of the sensor relative to the repositionable structure. Given the relationship and the objects and/or portions thereof that are recognized via machine learning or another computer vision technique, poses of one or more portions of the objects can be determined. The poses of one or more portions of objects can be determined in any technically feasible manner. In some embodiments where an object includes a repositionable structure (that is distinct from the repositionable structure of the computer-assisted system), and a portion of the object is a part of the repositionable structure, a pose of the portion of the object can be determined using kinematic data of the repositionable structure. In some embodiments, the poses of one more portions of objects can be determined using any suitable sensor data for locating and registering components relative to one another.

At process 504, the poses of one or more portions of the computer-assisted system (e.g., follower device 104) are determined in the reference frame. The one or more portions of the computer-assisted system can be selected in any technically feasible manner. In some embodiments, the one or more portions of the computer-assisted system can be selected based on defaults, a current configuration of the computer-assisted system with respect to objects and/or portions thereof, an operating procedure, operator preference, etc. In some embodiments, the one or more portions of the computer-assisted system can be obtained by dividing the computer-assisted system into portions using a grid, a quad tree, an octree, and/or other partitioning mechanisms.

In some embodiments, poses of the portion(s) of the computer-assisted system can be determined at process 504 based on kinematic data, one or more kinematic models of the computer-assisted system, and/or a 3D model of computer-assisted system, similar to the description above in conjunction with process 502. The poses of the portion(s) of the computer-assisted system can be determined in any technically feasible manner. In some embodiments, the pose of a portion of the computer-assisted system can be determined via machine learning and/or other computer vision techniques. As another example, in some embodiments, the pose of a portion of the computer-assisted system can be determined using any suitable sensor data, including sensor data for locating and registering components relative to one another. As yet another example, the pose of a portion of the computer-assisted system can be determined using any combination of the following types of sensors: joint encoders, inertial measurement units or IMUs on links, optical tracking, electromagnetic tracking, and thermal imaging. Although process 504 is described with respect to the pose of a portion of a computer-assisted system for simplicity, in some embodiments, the poses of any number of portions of a computer-assisted system can be determined with respect to the poses of any number of portions of objects. Alternatively or in addition, object(s) and the computer-assisted system (and/or portions thereof) can also be registered to each other so that the poses of the portion(s) of the computer-assisted system can be determined relative to the object(s).

At process 506, potential collisions between the one or more portions of the computer-assisted system and the one or more portions of objects are predicted based on the poses determined at processes 502 and 504. In some embodiments, rays are traced in the reference frame, described above in conjunction with processes 502 and 504, from points on the one or more portions of the computer-assisted system, which can be represented by a virtual 3D model, towards representations of the objects. For example, the representation of the objects can be point cloud representations generated by fusing sensor data from multiple sensors having different perspectives and/or different types of sensors, such as fusing color image data from imaging devices mounted at different locations on the computer-assisted system, fusing depth data from a LIDAR sensor and color image data from imaging device(s), etc. When rays are traced from points on the one or more portions of the computer-assisted system towards the representations of objects, the rays can be projected from each of the one or more portions of the computer-assisted system along a current trajectory of the computer-assisted system and/or a particular direction relative to the reference frame. When a traced ray intersects a portion of an object, the intersection corresponds to a potential collision. In some embodiments, in addition to predicting potential collisions between one or more portions of the computer-assisted system and one or more portions of objects, potential collisions between objects and/or portions thereof can also be predicted. In some examples, the traced ray can be in the direction of motion of a portion of the repositionable structure. In some embodiments, in lieu of a traced ray, a curved line can be projected from one or more portions of the computer-assisted system along a curved trajectory of the computer-assisted system and/or a particular instantaneous direction relative to a moving reference frame. In some embodiments, a curved line projection can be a circular arc with the instantaneous center of rotation of the computer-assisted system as it is maneuvered proximate to the patient.

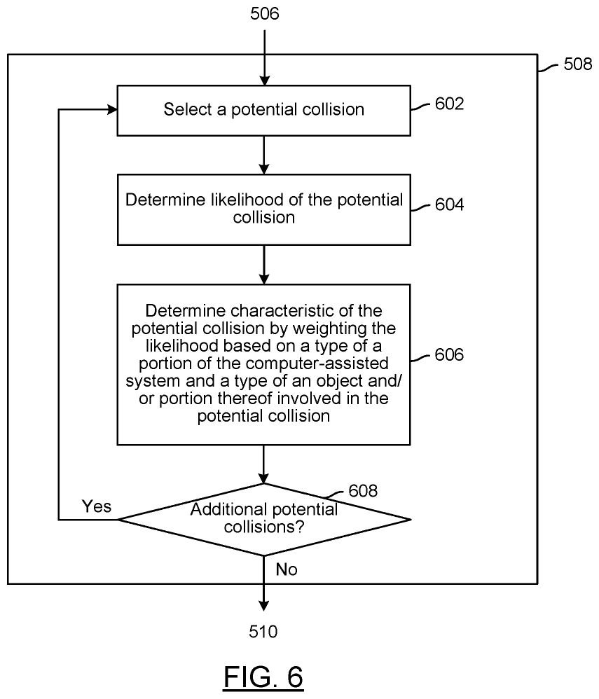

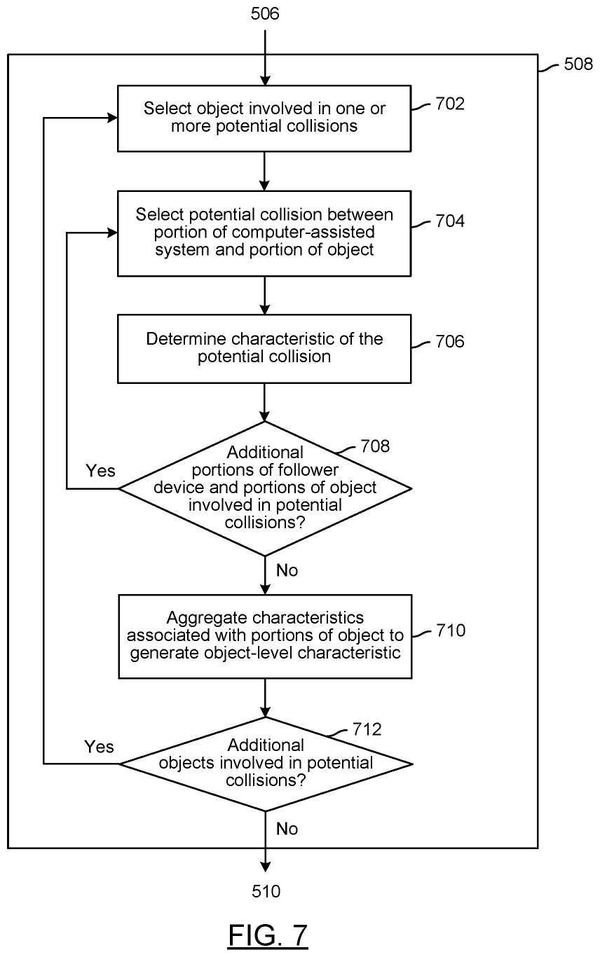

At process 508, one or more characteristics associated with each potential collision are determined. Examples of characteristics that can be used include a priority assigned to each potential collision, a likelihood of each potential collision, a time and/or distance to each potential collision, a safety-criticality of each potential collision, classifications of objects involved in each potential collision, whether a potential collision involves an operator-selected portion of the computer-assisted system and/or portion of an object, or a combination thereof. One or more characteristics (e.g., the priority assigned to each collision, the likelihood of potential collision, etc.) can be quantitative in some embodiments. One or more characteristics may not be represented quantitatively in some embodiments. One or more characteristics associated with each potential collision can be determined in any technically feasible manner. In some embodiments, a characteristic (e.g., a priority) can be determined for each potential collision by weighting a likelihood of the potential collision based on a type of the object or portion thereof and a type of the portion of the repositionable structure involved in the potential collision, as discussed in greater detail below in conjunction with FIG. 6. In some other embodiments, a characteristic (e.g., a priority) for each object can be determined by representing potential collisions of portions of the computer-assisted system with portions of the object using a tree, computing an intermediate characteristic for each portion of the object, and assuming the object is associated with multiple potential collision points, normalizing and aggregating the intermediate characteristics to determine a characteristic at the object level in the tree, as discussed in greater detail below in conjunction with FIG. 7.

In some embodiments, different characteristics can be determined for imaging devices and/or displays that are mounted differently. For example, a displayed view that is based on images captured by an imaging device (e.g., imaging device 202-1) that is mounted relatively high on the computer-assisted system can prioritize a patient on a table as an object of interest. In such cases, higher priorities and/or other characteristics can be assigned to potential collisions with the patient. As another example, a displayed view that is based on images from an imaging device (e.g., imaging device 202-4) that is mounted relatively low on the computer-assisted system can prioritize objects on or near the floor (e.g., the feet of an operator, a base of a platform or stand) as objects of interest. In such cases, higher priorities and/or other characteristics can be assigned to potential collisions with the object on or near the floor. As a further example, a view that is displayed via a head-mounted display can prioritize objects that are either not visible to an operator wearing the head-mounted device or that are not within the field of view of the head-mounted device.

In some embodiments, different characteristics can be determined for different types of sensors. Examples of different types of sensors include 2D RGB (red, green, blue) cameras, time-of-flight sensors, LIDAR sensors, etc. For example, a displayed view that is based on images captured by a particular type of sensor can prioritize certain types of objects as objects of interest when determining the characteristics.

In some embodiments, one or more characteristics can be based on a detected/tracked motion of the computer-assisted system. In some embodiments, a current or tracked motion of the computer-assisted system and a current trajectory of the repositionable system can be used to determine a priority for different objects. For example, potential collisions with objects that are within a threshold proximity to the detected/tracked trajectory can be assigned higher priorities. It should be noted that some objects within the threshold proximity of the predicted trajectory can be outside a field of view of an imaging device that captures images used to display XR indications of potential collisions. In some examples, objects outside the field of view of an imaging device can be detected and localized by employing known techniques such as simultaneous localization and mapping (SLAM).

FIG. 6 illustrates in greater detail process 508 of method 500 of FIG. 5, according to various embodiments. One or more of the example processes 602-608 can be implemented, at least in part, in the form of executable code stored on non-transitory, tangible, machine readable media that when run by one or more processors (e.g., the processor 150 in control system 140) can cause the one or more processors to perform one or more of the processes 602-608. In some embodiments, fewer or additional processes, which are not shown, can be performed. In some embodiments, one or more of the processes 602-608 can be ordered differently than shown in FIG. 6. In some embodiments, one or more of the processes 602-608 can be combined or performed simultaneously. In some embodiments, one or more of the processes 602-608 can be performed, at least in part, by one or more of the modules of control system 140, such as control module 170.

As shown, at process 602, one of the potential collisions predicted during process 506 is selected for processing.

At process 604, a likelihood of the potential collision is determined. The likelihood of the potential collision can be determined in any technically feasible manner. In some embodiments, a likelihood of the potential collision can be determined based on a time and/or a distance to the potential collision in at least one direction of interest. In such cases, potential collisions that are projected to occur sooner in time or between a portion of the computer-assisted system and a portion of an object that are closer together in a direction of interest can be assigned higher likelihoods. Conversely, potential collisions that are projected to occur later in time or between a portion of the computer-assisted system and a portion of an object that are farther apart in a direction of interest can be assigned lower likelihoods. The distance and direction of interest can be measured in any number of linear and/or rotational degrees of freedom (DOFs), including up to 6 DOFs. In some examples, the distance and/or direction of interest may be measured in the direction of relative motion between a portion of the computer-assisted system and a portion of an object. In some embodiments, a likelihood of the potential collision can be determined based on a historical frequency of the potential collision. In such cases, potential collisions that tend to occur more frequently can be assigned higher likelihoods. Conversely, potential collisions that tend to occur less frequently can be assigned lower likelihoods. In some embodiments, a likelihood of the potential collision can be determined based on a combination of factors, such as a time and/or a distance to the potential collision, as well as a historical frequency of the potential collision.

At process 606, a characteristic of the potential collision is determined by weighting the likelihood based on a type of a portion of the computer-assisted system and a type of a portion of an object associated with the potential collision. The weighted likelihood indicates a priority (i.e., a characteristic) associated with the potential collision. In other embodiments, characterstic(s) of potential collisions can be determined as the likelihood of the potential collisions, without weighting, or based on the time and/or distance to the potential collisions, the safety-criticality of the potential collisions, classifications of objects involved in the potential collisions, etc., as described above in conjunction with process 508.

For example, in the medical context, weight values can be assigned to objects and portions thereof in the following order, from highest to lowest: patients or other materials in a workspace to be manipulated (highest weight value), an operating table, personnel in or near the workspace, equipment that is susceptible to a high-risk of damage such as anesthesia monitoring equipment, lighting fixtures, a mayo stand and back table, and other equipment such as a vision cart and equipment that is susceptible to a low-risk of damage (lowest weight value). In addition, weight values can be assigned to portions of a computer-assisted system in the following order, highest to lowest: cannula mounts or any other portion of the computer-assisted system that is closest to objects of interest (i.e., that are associated with a potential initial point of collision and are assigned a highest weight value), other lowest/furthest extended portions of the computer-assisted system, such as a base of a repositionable structure of the computer-assisted system (assigned a lowest weight value). In some embodiments, an assigned weight value can account for the pairing of the portion of the computer-assisted system and the portion of the object that are involved in the potential collision. For example, when a potential collision between a particular portion of an object and a particular portion of the computer-assisted system is a significant safety issue or occurs frequently, a higher weight value can be assigned to the potential collision. In some embodiments, the weight values can be dynamically updated while the computer-assisted system is in motion. For example, the weight values can be updated based on changing factors such as whether an object is in the field of view of an imaging device. In such cases, the objects that are of interest and associated with higher weight values can change as the computer-assisted system is in motion. In some embodiments, the weight values can be set at a point in time (e.g., in response to a user input before moving the computer-assisted system) and remain static while the computer-assisted system is in motion. In some embodiments, the weight values can account for operator preference. For example, an operator can indicate (e.g., via a touchscreen or other input device) a target object for collision avoidance or a target position to achieve. In such cases, the weight values can be set or reset based on the operator preferences that are input. In some examples, the weight values can be set based on safety-criticality of the type of collision, whereas in other examples, the weight values may be set based on the historical frequency of the type of collision.

At process 608, when there are additional potential collisions, process 508 returns to process 602, where another potential collision is selected for processing. Alternatively, when there are no additional potential collisions, method 500 continues to process 510, discussed in greater detail below.

FIG. 7 illustrates in greater detail process 508 of method 500 of FIG. 5, according to various other embodiments. Example processes 702-712 are alternatives that can be performed in lieu of processes 602-608, described above in conjunction with FIG. 6, to determine the characteristic(s) associated with each potential collision at process 508. One or more of processes 702-712 can be implemented, at least in part, in the form of executable code stored on non-transitory, tangible, machine readable media that when run by one or more processors (e.g., the processor 150 in control system 140) can cause the one or more processors to perform one or more of the processes 702-712. In some embodiments, fewer or additional processes, which are not shown, can be performed. In some embodiments, one or more of the processes 702-712 can be ordered differently than shown in FIG. 7. In some embodiments, one or more of the processes 702-712 can be combined or performed simultaneously. In some embodiments, one or more of the processes 702-712 can be performed, at least in part, by one or more of the modules of control system 140, such as control module 170.

As shown, at process 702, one of the objects that is predicted to be involved in a potential collision during process 506 is selected for processing.

At process 704, a potential collision between a portion of the computer-assisted system and a portion of the object is selected for processing. In some embodiments, the object can be divided into portions using a grid, a quad tree, an octree, and/or other partitioning mechanisms. Similarly, the computer-assisted system can be divided into portions using another grid, quad tree, octree, and/or other partitioning mechanisms. In such cases, the potential collision can be between one of the portions of the objects and one of the portions of the computer-assisted system.

At process 706, an intermediate characteristic of the potential collision is determined. The intermediate characteristic can be determined in any technically feasible manner. In some embodiments, the intermediate characteristic can be a priority determined as a weighted likelihood of the potential collision according to processes 602-608, described above in conjunction with FIG. 6. In some other embodiments, the intermediate characteristic can be determined in any technically feasible manner, such as a likelihood of the potential collisions or based on a time and/or distance to the potential collision, a safety-criticality of the potential collision, a classification of objects involved in the potential collisions, etc., as described above in conjunction with process 508. In some embodiments, the intermediate characteristic can be stored in a leaf node of a tree. For example, each leaf node can be associated with a different portion of the object.

At process 708, when additional portions of the computer-assisted system (e.g., follower device 104) and portions of the object are involved in potential collisions, process 508 returns to process 704, where another potential collision between a portion of the computer-assisted system and a portion of the object is selected for processing.

Alternatively, when no additional portions of the computer-assisted system and portions of the object are involved in potential collisions, process 508 continues to process 710, where the intermediate characteristics associated with different portions of the object are aggregated to determine an object-level characteristic. In some embodiments, aggregating the intermediate characteristics can include normalizing the intermediate characteristics stored in leaf nodes of a tree and adding the normalized intermediate characteristics together to obtain a characteristic at the object level in the tree.

At process 712, when additional objects are involved in potential collisions, the process 508 returns to process 702, where another object that is involved in one or more potential collisions is selected for processing.

Returning to FIG. 5, at process 510, a subset of the potential collisions is selected based on the one or more characteristics associated with each potential collision. Depending on the characteristics, potential collisions can be selected in any technically feasible manner. In some embodiments, the subset of potential collisions is selected according to a predefined rule. The rule can require one or more characteristics associated with a potential collision to satisfy one or more criteria in order for the potential collision to be selected. For example, a rule can require potential collisions with certain types of objects from a list of objects (e.g., patients or other humans) that is predefined (e.g., either as a default or specified by an operator) to be selected. In such a case, the characteristic can be the classification of the object associated with the potential collision, and the classification can be compared with the list of objects to determine if the rule is satisfied. As another example, the subset of potential collisions that are selected can include potential collisions between a set of points on the computer-assisted system that are predefined and objects in the physical environment. In such cases, a rule can require that each potential collision be between one of the set of points (the characteristic) and objects in the environment in order to be selected. For example, the set of points can be a set of lowest points, such as the distal portions (e.g., cannula mounts) of manipulator arms of a repositionable structure.

In some embodiments, the subset of potential collisions is selected by (1) computing a score based on one or more characteristics, and (2) selecting potential collisions associated with scores that satisfy one or more criteria. For example, a criterion can require that a characteristic, or a score that is computed as a function of multiple characteristics, have a value that exceeds a threshold, be among a number (e.g., the top 10 percent) of highest values, be among a number (e.g., the bottom 10 percent) of lowest values, or be within a range of values, in order for a potential collision that is associated with the characteristic or function of multiple characteristics to be selected. The threshold, number of highest values, number of lowest values, or range of values can be predefined (e.g., as a default or according to specification by an operator). As a specific example, when the characteristic is a priority value, such as the weighted likelihood described above in conjunction with FIG. 6, a given number of potential collisions having the highest priorities can be selected. As another example, when the characteristic is a likelihood, a given number of potential collisions having the highest likelihoods can be selected.

In some embodiments, the subset of potential collisions is selected using a decision tree. For example, multiple characteristics associated with a potential collision (e.g., a certain portion of computer-assisted system, a certain type of object, a direction of motion, a speed, a likelihood of collision, etc.) can be determined. Then, the decision nodes in a tree can be traversed based on values of the multiple characteristics to arrive at a decision on whether or not to select the potential collision.

In some embodiments, intermediate characteristics are determined for different portions of each object and aggregated to determine an object-level characteristic for the object. In such cases, the subset of potential collisions can be selected based on object-level characteristics that are determined for different objects. In some examples, the object-level characteristics for different objects can be sorted in any technically feasible manner, and a subset of potential collisions with those objects can be selected based on the ranking of the corresponding object-level characteristics in the sorted list.

For example, assume that the following potential collisions have been predicted at process 506: top of an instrument assembly (or last link of a follower device) to an imaging cart (potential collision 1); top of the follower device to a ceiling light fixture (potential collision 2); cannula mount on an arm of the follower device with a patient on an operating table (potential collision 3). In such a case, the following ordered pairs of (time to collision, safety-criticality) could be determined as the characteristic for each potential collision at process 508: (5 seconds, “medium”) for potential collision 1, (5 seconds, “low”) for potential collision 2, and (30 seconds, “high”) for potential collision 3. Then, at process 510, the characteristics can be sorted based on time to collision (i.e., 5 seconds, 5 seconds, and 30 seconds for potential collision 1, potential collision 2, and potential collision 3, respectively), as well as based on safety-criticality (i.e., “low,” “medium,” and “high” for potential collision 2, potential collision 1, and potential collision 3, respectively). In addition, a decision tree can be traversed in which a first node of the decision tree selects the potential collision(s) with the lowest time to collision (potential collisions 1 and 2 in the example), and a second node of the decision tree selects the potential collision(s) associated with the highest safety-criticality out of the potential collision(s) with the lowest time to collision (potential collision 1 in the example). As a result, potential collision 1 can be selected at process 510 as the subset of potential collisions to be displayed.

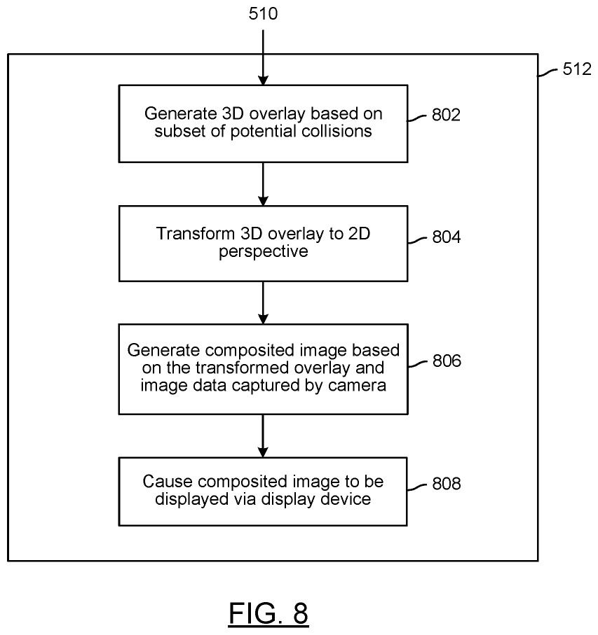

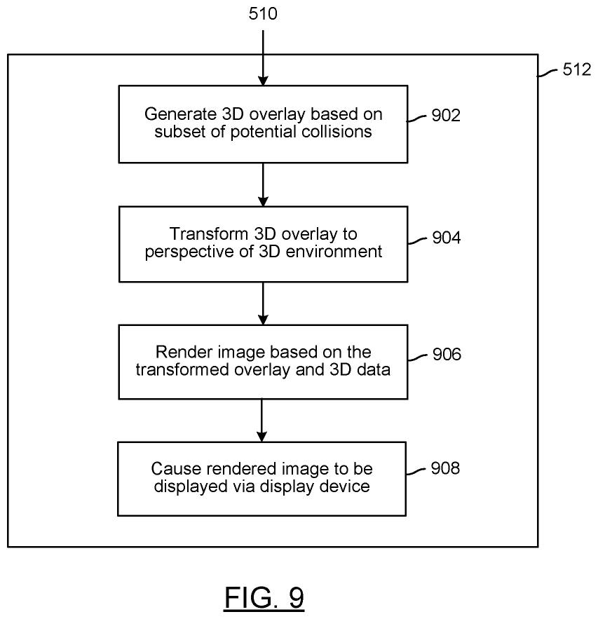

At process 512, one or more XR indications of the subset of potential collisions is caused to be displayed. In some embodiments, the one or more XR indications can include a 2D overlay for a 2D display that matches, or does not match, the image plane of an imaging device. The one or more XR indications can also be dynamically updated in real time according to changing environmental conditions. For example, displayed trajectories could be changed to account for a changing direction of motion of a computer-assisted system, additional trajectories could be displayed to indicate new potential collisions that are associated with characteristics satisfying certain criteria, trajectories for potential collisions that are associated with characteristics no longer satisfying certain criteria can no longer be displayed, etc. Processes for generating and displaying a 2D overlay are discussed in greater detail below in conjunction with FIG. 8. In some embodiments, the one or more XR indications can include a 3D overlay that is rendered in a 3D virtual environment. Processes for generating and displaying a 3D overlay are discussed in greater detail below in conjunction with FIG. 9.

Various XR indications can be displayed. In some embodiments, an XR indication can include geometrical abstractions. For example, the geometrical abstractions can include virtual laser lines that are parallel to a surface on which the computer-assisted system is located and indicate intersections between portions of the computer-assisted system (e.g., cannula mounts or other portions most likely to collide with objects) and objects in the physical environment (e.g., a draped patient on an operating room table). As another example, the geometrical abstractions can include a cross hair with a placement tolerance to indicate where a portion of the computer-assisted system will end up if the computer-assisted system is moved in a straight line along a current trajectory. In yet another example, the geometrical abstractions can include a surface or a plane of significance, such as a plane tangent to the enclosing volume of a region on a patient profile at a highest point of a volume of the patience referenced to the floor. In some embodiments, an XR indication can include one or more trajectories and/or a target position of the computer-assisted system. In some embodiments, an XR indication can include indications of allowed directions of motion of a portion of the computer-assisted system. For example, the XR indication can include a current trajectory, a desired trajectory, a target position of the computer-assisted system, an allowed direction of motion, and/or a tolerance that are based on operator preference, target object(s) to avoid or approach, etc. The trajectories can include straight lines based on current directions of motion of portions of the computer-assisted system. Additionally or alternatively, the trajectories can include curved or other types of lines to indicate trajectories of portions of the computer-assisted system that are not moving along straight lines. In some embodiments, the trajector(ies) and the target position, and/or other information, can be projected onto the floor in a 2D or 3D overlay. In some embodiments, the XR indication can include an indication of a virtual fixture implemented in order to guide the wheels of the computer-assisted system to move along certain pre-defined directions while constraining the motion of the computer-assisted system in certain other directions to prevent impending collisions.

In some embodiments, an XR indication can include indications of recommended adjustments to be made manually or that can be made automatically. In such cases, the recommended adjustments can be represented using arrows, animations, or any other technically feasible manner. For example, the recommended adjustments can include an adjustment to the vertical height of a repositionable structure of the computer-assisted system. As another example, the recommended adjustments can include an adjustment to a portion (e.g., a single manipulator arm) of the repositionable structure. In some embodiments, an XR indication can include text and/or animations. For example, the text and/or animations can indicate clearances (e.g., clearance of a patient, lighting fixture, and/or other objects), distances, warnings (e.g., of specific potential collisions, to stop or slow down, etc.), whether and/or how a potential collision is resolvable (e.g., by repositioning a repositionable structure of the computer-assisted system or the entire computer-assisted system, by repositioning the object, etc.), how to maneuver the computer-assisted system and/or reconfigure the repositionable structure of the computer-assisted system to avoid a potential collision, etc.

In some embodiments, an XR indication can include indications of an area of a physical environment, a range of motion, a kinematic workspace, and/or workspace of a repositionable structure of the computer-assisted system. For example, an XR indication can indicate an area of the physical environment to approach or avoid. In some embodiments, an XR indication can include indications of state changes (e.g., ON or OFF), such as changes in a state of the computer-assisted system and/or changes in a state of the physical environment. In such cases, the state changes can be indicated, e.g., by changes in color or using text. For example, current trajectory lines can turn yellow or red when a current trajectory of the computer-assisted system deviates from a desired trajectory. As another example, warnings and/or changes in color of overlay elements can be used to indicate a higher risk or imminence of collision. As a specific example, the current trajectory lines can change from green to yellow, and then from yellow to red as the imminence of collision increases. As another example, the current trajectory lines can change from not flashing to flashing when the imminence of collision increases beyond a threshold. In some embodiments, an XR indication can include 2D or 3D avatars, icon representations, or generic renderings of detected objects, such as operators, fixtures, etc. In such cases, the object may or may not be in the current field of view of an imaging device that captures images on which the XR indication is overlaid. Objects and/or potential collisions that are not in the field of view can be indicated in any technically feasible manner. For example, text, arrows, and/or a combination thereof around the border of a displayed image can be used to indicate off-screen objects that the computer-assisted system can potentially collide with. In some embodiments, the XR indication can include an indication of a measurement of angular or linear distance between at least a portion of the computer-assisted system and at least a portion of an object of interest. In some embodiments, an XR indication can indicate multiple potential collision points. For example, if an operator walks between an object and the computer-assisted system, lines may be displayed that continue “through” the operator and indicate both a potential collision of the computer-assisted system with the operator and a potential collision of the computer-assisted system with the object.

FIG. 8 illustrates in greater detail process 512 of method 500 of FIG. 5, according to various embodiments. One or more of the example processes 802-808 can be implemented, at least in part, in the form of executable code stored on non-transitory, tangible, machine readable media that when run by one or more processors (e.g., the processor 150 in control system 140) can cause the one or more processors to perform one or more of the processes 802-808. In some embodiments, fewer or additional processes, which are not shown, can be performed. In some embodiments, one or more of the processes 802-808 can be ordered differently than shown in FIG. 8. In some embodiments, one or more of the processes 802-808 can be combined or performed simultaneously. In some embodiments, one or more of the processes 802-808 can be performed, at least in part, by one or more of the modules of control system 140, such as control module 170.

As shown, at process 802, a 3D overlay is generated based on a subset of potential collisions. In some embodiments, the 3D overlay can indicate the subset of potential collisions determined during process 510. In some embodiments, the 3D overlay can additionally indicate other information. In some embodiments, the 3D overlay, or a portion thereof, can be locked to a desired reference frame. For example, the 3D overlay can be locked to a local reference frame associated with the computer-assisted system. In such cases, the 3D overlay can move along with the computer-assisted system. As another example, the 3D overlay can be locked to a global reference frame. In such cases, the 3D overlay does not move along with the computer-assisted system. In some other embodiments, the 3D overlay, or a portion thereof, can be not locked to any reference frame. For example, the 3D overlay can move along with movement of a sensor providing sensor data used to determine the information in the 3D overlay. In other examples, the 3D overlay can move or change appearance based on system state or event data from the computer-assisted system.

At process 804, the 3D overlay is transformed to a 2D perspective associated with the view of an imaging device. The 3D overlay is transformed to a 2D overlay for a 2D display that matches (or, alternatively, does not match) an image plane of the imaging device. In some embodiments, intrinsic and/or extrinsic properties of the imaging device can be used to transform the 3D overlay according to well-known techniques. In such cases, the 3D overlay can be projected onto an image plane that is determined based on intrinsic and/or extrinsic properties of the imaging device. Examples of intrinsic properties of an imaging device include optical, geometric, and digital parameters (e.g., zoom, pan, etc.) of the imaging device including focal length, scaling, image center, camera lens distortions in image data captured by the imaging device, etc. An example of extrinsic properties includes a relative pose of the imaging device to the rest of the computer-assisted system.

At process 806, a composited image is generated based on the transformed overlay and image data captured by the imaging device. In some embodiments, the composited image is an XR image. Although described herein primarily with respect to generating a composite, in some embodiments, a perspective-corrected view of XR content can be displayed without combining the XR content with image data.

At process 808, the composited image is caused to be displayed via a display device (e.g., display device 302-1 or 302-2). Any technically feasible 2D or 3D display device can be used in some embodiments, and the display device can be placed at any suitable location. In some embodiments, one or more display devices can be located at a user control interface (helm) (e.g., user control interface 304) of the computer-assisted system. As another example, in some embodiments, one or more display devices can be included in a handheld device or a head-mounted device. In some embodiments, the display devices also include touch screens to allow user-interaction with the system, such as (a) selecting which XR indications to display, (b) selecting the frame of reference to display the XR indications in, (c) indicating a target object to monitor for collisions, (d) indicating a target position of the base of the a computer-assisted device based on an operator preference according to safety considerations for collision with the base of an object (e.g., an operating table), (e) selecting objects or points to measure angular or linear distance between, and/or (f) commanding the motion of at least a portion of the computer-assisted system, system state change, or event in response to the information displayed on the display device.

Although process 808 is described with respect to a single display device, in some embodiments, a composited image, or different composited images, can be displayed via one display device or multiple display devices. For example, one display device (e.g., display device 302-1 or 302-2) can be focused on a floor and display an overlay that is projected on the floor, while another display device (e.g., the other display device 302-1 or 302-2) can be focused on the height of a workspace or patient and display a corresponding overlay. In such cases, the same or different characteristics can be used to select potential collisions to indicate on the different display devices. As another example, overlays can be displayed along with different video feeds. In some embodiments, composited images are displayed to different operators who can interact with different user interfaces, and a handshake mechanism is used to arbitrate between inputs by the operators. For example, one user interface that is displayed via a display device can permit an operator on a non-sterile side of the computer-assisted system to command motion of a portion of the computer-assisted system on the sterile side, while another user interface that is displayed via another display device can permit a different operator on the sterile side to command motion of the same portion or a different portion of the computer-assisted system.

FIG. 9 illustrates in greater detail process 512 of method 500 of FIG. 5, according to other various embodiments. Example processes 902-908 are alternatives that can be performed in lieu of processes 802-808 to cause the XR reality indication of a subset of potential collisions to be displayed at process 512. One or more of the processes 902-908 can be implemented, at least in part, in the form of executable code stored on non-transitory, tangible, machine readable media that when run by one or more processors (e.g., the processor 150 in control system 140) can cause the one or more processors to perform one or more of the processes 902-908. In some embodiments, fewer or additional processes, which are not shown, can be performed. In some embodiments, one or more of the processes 902-908 can be ordered differently than shown in FIG. 9. In some embodiments, one or more of the processes 902-908 can be combined or performed simultaneously. In some embodiments, one or more of the processes 902-908 can be performed, at least in part, by one or more of the modules of control system 140, such as control module 170.

As shown, at process 902, a 3D overlay is generated based on a subset of potential collisions. Process 902 can be similar to process 802, described above in conjunction with FIG. 8, in some embodiments.

At process 904, the 3D overlay is transformed to the perspective of a 3D virtual environment. In some embodiments, the 3D overlay can be transformed to the perspective of a 3D virtual environment according to well-known techniques. For example, the 3D overlay can be translated, rotated, and/or scaled based on the placements of representations of the computer-assisted system and objects within the 3D virtual environment, and a scale associated with the 3D virtual environment.

At process 906, an image is rendered based on the transformed overlay and 3D data. In some embodiments, the 3D data can include point cloud data acquired by various sensors. In such cases, fused point cloud data from multiple sensor devices can be rendered along with the 3D overlay in a 3D virtual environment. In some examples, an abstracted representation of the point cloud data may be displayed, such as a surface mesh or convex hull or 3D model reprentations of segmented portions of the point cloud.

At process 908, the rendered image is caused to be displayed via a display device (e.g., display device 302-1 or 302-2). Process 908 is similar to process 808, described above in conjunction with FIG. 8.

Although described herein primarily with respect to display devices that provide 2D or 3D views, in some embodiments, a 3D or 2D overlay can be projected into a physical space. For example, one or more laser beams could be emitted toward points on objects that are associated with a subset of potential collisions.

Returning to FIG. 5, subsequent to process 512, assuming that the computer-assisted system is still being repositioned, method 500 returns to process 502.

FIGS. 10A-10B illustrate example 2D visual guidance displays, according to various embodiments. As shown in FIG. 10A, a display 1000 includes an image of a worksite captured by an imaging device (e.g., imaging device 202-1) that is mounted relatively high on a computer-assisted system (e.g., follower device 104). In addition, display 1000 includes a 2D AR overlay that (1) highlights an object of interest, shown as an operating table 1005, and (2) indicates a subset of potential collisions between the computer-assisted system and operating table 1005. Display 1000 can be displayed on one of display devices 302-1 or 302-2, or in any other technically feasible manner. Illustratively, the AR overlay includes lines 1002, 1004, 1006, and 1008 associated with each of the cannula mounts of the computer-assisted system, which can be the lowest points (e.g., the distal portions) on arms of the computer-assisted system. Lines 1002, 1004, 1006, and 1008 show projections from points corresponding to the cannula mounts to corresponding points on the operating table 1005, at which the cannula mounts are projected to collide with the operating table. Poses of portions of operating table 1005 and portions of the computer-assisted system in a common reference frame can be determined according to processes 502-504, described above in conjunction with FIG. 5. Lines 1002, 1004, 1006, and 1008 correspond to rays that are projected from the points corresponding to the cannula mounts along a current trajectory of the computer-assisted system to operating table 1005, as described above in conjunction with process 506. In addition, lines 1002, 1004, 1006, and 1008 can be associated with a subset of potential collisions (with operating table 1005 and/or other objects) that are selected based on one or more characteristics of the potential collisions, as described above in conjunction with processes 508-510.

The AR overlay in display 1000 further includes text labels 1010, 1012, 1014, 1016 indicating identifiers (IDs) associated with cannula mounts whose positions are being projected using the lines 1002, 1004, 1006, and 1008, respectively. In addition, the AR overlay includes lines 1018 corresponding to a current trajectory of the center of an orienting platform (e.g., center 201 of orienting platform 204) of computer-assisted system, and a cross-hair 1020 that indicates a placement tolerance for guiding the center of the orienting platform to a target point on or near operating table 1005.

As shown in FIG. 10B, a display 1030 includes an image of a worksite captured by an imaging device (e.g., imaging device 202-4) that is mounted relatively low on a computer-assisted system. Display 1030 can be displayed on one of display devices 302-1 or 302-2, or in any other technically feasible manner. Display 1030 can be presented alone or together (e.g., on the same display device or a separate display device) with display 1000. Illustratively, display 1000 includes a 2D overlay that (1) highlights operating table 1005, and (2) indicates a current trajectory 1032 of computer-assisted system as well as lines 1034 indicating the trajectory of an orienting platform, which correspond to lines 1018, described above in conjunction with FIG. 10A. Poses of portions of operating table 1005 can be determined according to process 504, described above in conjunction with FIG. 5. Current trajectory 1032 and lines 1018 can be determined based on a change in pose of the computer-assisted system and the orienting platform over time. As shown, current trajectory 1032 and lines 1034 have been projected onto a 2D plane corresponding to a floor in the image captured by the imaging device. The width of the current trajectory 1032 can correspond to the width of the base of the computer-assisted system and the distal horizontal line can indicate where the trajectory might intersect with the operating table 1005. The width of the line 1018 can correspond to the placement tolerance described above.

FIG. 11 illustrates an example 3D visual guidance display 1100, according to various embodiments. As shown, display 1100 includes a rendered image 1110 of a 3D virtual environment corresponding to a physical environment, as well as a captured image 1112 of the physical environment. Display 1100 can be displayed on one of display devices 302-1 or 302-2, or in any other technically feasible manner. Rendered image 1110 includes a representation of a computer-assisted system 1102 and representations of objects in the physical environment. Illustratively, the representations of objects include a point cloud representation of an operating room scene 1100 as viewed from a camera mounted on the computer-assisted system. In some embodiments, point cloud data can be generated by fusing image data from multiple imaging devices, or in any other technically feasible manner. In some examples, the multiple imaging devices may not all be mounted on the computer-assisted system and may be registered to the computer-assisted system instead. Poses of portions of operating table 1106 and portions of computer-assisted system 1102 in a common reference frame can be determined according to processes 502-504, described above in conjunction with FIG. 5. In addition, rendered image 1110 includes a 3D overlay 1104 indicating portions of computer-assisted system 1102 that are predicted to collide with one or more of the objects. In some embodiments, 3D overlay 1104 can be created to indicate a subset of potential collisions that are selected based on one or more characteristics associated with the potential collisions, as described above in conjunction with processes 506-510 of FIG. 5. 3D overlay 1104 can then be rendered, along with the representations of computer-assisted system 1102 and other objects (e.g., operating table 1106), in a 3D virtual environment to generate rendered image 1110, according to processes 902-908 of FIG. 9.

Although example visual guidance displays are described above with respect to FIGS. 10A-B and 11 for illustrative purposes, other types of visual guidance displays, such as visual guidance displays that include the XR indications described above in conjunction with FIG. 8, are also contemplated.

Advantageously, techniques are disclosed that enable a computer-assisted system to be repositioned at a target position and/or orientation relative to a worksite while avoiding obstacles in the vicinity of the worksite. The disclosed techniques can decrease the likelihood that collisions with obstacles occur while also reducing the time needed to reposition the computer-assisted system at the target position and/or orientation.

Although illustrative embodiments have been shown and described, a wide range of modification, change and substitution is contemplated in the foregoing disclosure and in some instances, some features of the embodiments may be employed without a corresponding use of other features. One of ordinary skill in the art would recognize many variations, alternatives, and modifications. Thus, the scope of the invention should be limited only by the following claims, and it is appropriate that the claims be construed broadly and, in a manner, consistent with the scope of the embodiments disclosed herein.

1. A computer-assisted system comprising:
a sensor system configured to capture sensor data of an environment; and
a control system communicably coupled to the sensor system,
wherein the control system is configured to:
determine a pose of a portion of an object in the environment based on the sensor data, the pose of the portion of the object comprising at least one parameter selected from the group consisting of: a position of the portion of the object and an orientation of the portion of the object,
determine a pose of a portion of the computer-assisted system, the pose of the portion of the computer-assisted system comprising at least one parameter selected from the group consisting of: a position of the portion of the computer-assisted system and an orientation of the portion of the computer-assisted system,
determine at least one characteristic associated with a potential collision between the portion of the object and the portion of the computer-assisted system based on the pose of the portion of the object and the pose of the portion of the computer-assisted system,
select the potential collision for display based on the at least one characteristic, and
cause an extended reality (XR) indication of the potential collision to be displayed to an operator via a display system.

2. The computer-assisted system of claim 1, wherein to select the potential collision, the control system is configured to:
determine that a first characteristic included in the at least one characteristic satisfies one or more criteria,
wherein the first characteristic is based on at least one parameter selected from the group consisting of: a priority associated with the potential collision, a likelihood of the potential collision, a time to the potential collision, a distance to the potential collision, a measure of safety associated with the potential collision, a type of the object, a type of the portion of the object, a type of the portion of the computer-assisted system, a frequency of collisions between the type of the object and a type of the computer-assisted system, a frequency of collisions between the type of the portion of the object and the type of the portion of the computer-assisted system, a distance between the portion of the object and the portion of the computer-assisted system, a configuration of the computer-assisted system, a preference of the operator, an operating procedure, a direction of relative motion between portion of the object and the portion of the computer-assisted system, and whether the portion of the object is within a field of view of the sensor system.

3-4. (canceled)

5. The computer-assisted system of claim 1, wherein to select the potential collision, the control system is configured to:
compute a score value based on a function of the at least one characteristic; and
determine that the score value satisfies one or more criteria.

6. The computer-assisted system of claim 1, wherein to select the potential collision, the control system is configured to:
process the at least one characteristic using a decision tree.

7. The computer-assisted system of claim 1, wherein to select the potential collision, the control system is configured to:
select the potential collision further based on a predefined point associated with the portion of the computer-assisted system.

8. The computer-assisted system of claim 1, wherein the XR indication of the potential collision indicates an intersection between the portion of the object and a projected position of the portion of the computer-assisted system in the environment.

9. (canceled)

10. The computer-assisted system of claim 1, wherein the XR indication comprises at least one indication selected from the group consisting of: a geometrical indication, an indication of a trajectory of the computer-assisted system, an indication of a potential collision point, a target position indication, an indication of a recommended adjustment to the computer-assisted system, an indication of a range of motion or workspace of a repositionable structure, an indication of a state of the computer-assisted system, an indication of a state of an environment, an indication of a target position for a first portion of the computer-assisted system, an indication of a tolerance in positioning the first portion of the computer-assisted system, an indication of allowed directions of motion of the computer-assisted system, a color indication, text, an animation, an avatar, an icon, a physical measurement, and a rendering of the object.

11. (canceled)

12. The computer-assisted system of claim 1, further comprising:
a helm that includes the display system,
wherein the helm is disposed on an opposite side of the computer-assisted system relative to a repositionable structure.

13-14. (canceled)

15. The computer-assisted system of claim 1, wherein the control system is further configured to:
determine the potential collision based on the post of the portion of the object and the portion of the computer-assisted system by tracing a ray from the portion of the computer-assisted system to the portion of the object in a reference frame.

16. The computer-assisted system of claim 15, wherein the ray is traced in a current direction of motion or a predicted direction of motion of the portion of the computer-assisted system.

17-19. (canceled)

20. The computer-assisted system of claim 1, wherein the control system is further configured to:
determine the at least one characteristic further based on one or more weight values that are assigned to at least one of the portion of the computer-assisted system or the portion of the object.

21-25. (canceled)

26. The computer-assisted system of claim 1, wherein to determine the at least one characteristic, the control system is configured to:
determine a plurality of intermediate characteristics that are associated with different pairs of portions of the object and portions of the computer-assisted system; and
aggregate the intermediate characteristics to determine the at least one characteristic.

27-28. (canceled)

29. A method comprising:
determining a pose of a portion of an object in an environment based on sensor data captured by a sensor system, the pose of the portion of the object comprising at least one parameter selected from the group consisting of: a position of the portion of the object and an orientation of the portion of the object;
determining a pose of a portion of a computer-assisted system, the pose of the portion of the computer-assisted system comprising at least one parameter selected from the group consisting of: a position of the portion of the computer-assisted system and an orientation of the portion of the computer-assisted system;
determining at least one characteristic associated with a potential collision between the portion of the object and the portion of the computer-assisted system based on the pose of the portion of the object and the pose of the portion of the computer-assisted system;
selecting the potential collision for display based on the at least one characteristic; and
causing an extended reality (XR) indication of the potential collision to be displayed to an operator via a display system.

30-34. (canceled)

35. The method of claim 29, wherein selecting the potential collision comprises:
selecting the potential collision further based on a predefined point associated with the portion of the computer-assisted system.

36-41. (canceled)

42. The method of claim 29, further comprising determining the potential collision based on the pose of the portion of the object and the pose of the portion of the computer-assisted system by tracing a ray from the portion of the computer-assisted system to the portion of the object in a reference frame.

43-46. (canceled)

47. The method of claim 29, further comprising determining the at least one characteristic further based on one or more weight values that are assigned to at least one of the portion of the computer-assisted system or the portion of the object.

48-52. (canceled)

53. The method of claim 29, wherein determining the at least one characteristic comprises:
determining a plurality of intermediate characteristics that are associated with different pairs of portions of the object and portions of the computer-assisted system; and
aggregating the intermediate characteristics to determine the at least one characteristic.

54-55. (canceled)

56. One or more non-transitory machine-readable media comprising a plurality of machine-readable instructions which when executed by one or more processors are adapted to cause the one or more processors to perform a method comprising:
determining a pose of a portion of an object in an environment based on sensor data captured by a sensor system, the pose of the portion of the object comprising at least one parameter selected from the group consisting of: a position of the portion of the object and an orientation of the portion of the object;
determining a pose of a portion of a computer-assisted system, the pose of the portion of the computer-assisted system comprising at least one parameter selected from the group consisting of: a position of the portion of the computer-assisted system and an orientation of the portion of the computer-assisted system;
determining at least one characteristic associated with a potential collision between the portion of the object and the portion of the computer-assisted system based on the pose of the portion of the object and the pose of the portion of the computer-assisted system;
selecting the potential collision for display based on the at least one characteristic; and
causing an extended reality (XR) indication of the potential collision to be displayed to an operator via a display system.

57. The one or more non-transitory machine-readable media of claim 56, wherein determining the potential collision comprises tracing a ray from the portion of the computer-assisted system to the portion of the object in a reference frame.

58. The one or more non-transitory machine-readable media of claim 56, wherein determining the at least one characteristic comprises:
determining a plurality of intermediate characteristics that are associated with different pairs of portions of the object and portions of the computer-assisted system; and
aggregating the intermediate characteristics to determine the at least one characteristic.
